(19) United States
(12) Patent Application Publication
(10) Pub. No.: US 2021/0319913 A1
(43) Pub. Date: Oct. 14, 2021

(54) SYSTEMS AND METHODS FOR OBTAINING AND MONITORING RESPIRATION, CARDIAC FUNCTION, AND OTHER HEALTH DATA FROM PHYSICAL INPUT

(71) Applicants: Giovanna Guidoboni, Chesterfield, MO (US); George Chronis, Chesterfield, MO (US); Samantha Kurkowski, Maryland Heights, MO (US)

(72) Inventors: Giovanna Guidoboni, Chesterfield, MO (US); George Chronis, Chesterfield, MO (US); Samantha Kurkowski, Maryland Heights, MO (US)

(21) Appl. No.: 17/228,249

(22) Filed: Apr. 12, 2021

Related U.S. Application Data
(60) Provisional application No. 63/008,396, filed on Apr. 10, 2020.

(51) Int. Cl. G16H 50/80; A61B 5/08; A61B 5/00

(52) U.S. Cl. CPC G16H 50/80; A61B 5/0816; A61B 5/6831; A61B 5/7235; A61B 5/6898; A61B 5/7282

(57) ABSTRACT

Systems and methods for the physical detection of respiration and cardiac function and particularly alteration in respiration which can be used to detect potential damage to the lungs, mucus accretion, or other symptoms which can be indicative of respiratory infection. This physical detection is typically performed using specialized simple sensors which are included as part of a bed or are attached to or placed on or under a mattress or, in an embodiment, using physical sensors in mobile devices such as smart phones allowing for much broader data collection, monitoring, and identification of those afflicted.

### CROSS REFERENCE TO RELATED APPLICATION(S)

This application claims the benefit of U.S. Provisional Patent Application Ser. No. 63/008,396, filed Apr. 10, 2020, the entire disclosure of which is herein incorporated by reference.

### BACKGROUND OF THE INVENTION

### Field of the Invention

The present disclosure relates to systems and methods for utilizing physical sensors in the detection of specific health related conditions. In particular, to the use of physical sensors in a mobile device to monitor respiration and cardiac function.

### Description of the Related Art

Motion sensors and detectors are already in use in a variety of medical areas. For example, sensors located in a patient's bed or on a chair can be used to sense when a patient has gotten up, is attempting to stand, or has not moved for a period of time. These kinds of sensors can be used to detect that a patient is currently at an increased risk of falling, issue warnings before possible falls occur, detect falls when they occur, and alert against pressure ulcers in both hospitals and senior care facilities.

In the wake of the 2020 COVID-19 virus pandemic, the human population saw the global spread of a deadly disease leading to mass “social distancing” and the use of respiratory masks in an attempt to halt its spread before vaccines could be made available. Social distancing was effectively a lighter form of quarantine where all individuals were intended simply to be kept at a distance from each other so that COVID-19, which was believed to be spread from an infected individual to others primarily by airborne transmission, did not pass from an infected person (who may not have been aware they were infected) to one that was not. Masks or other face coverings, which ranged from homemade sewn fabric constructions to advanced N95 particulate respirators, were also widely used to attempt to block airborne particulates ejected by one person from being inhaled by another.

The effectiveness of these measures is the subject of some debate. Diseases spread by airborne particles and respiration can be more difficult to contain than those that require other forms of transmission. For example, diseases spread by blood exchange, such as Acquired Immune Deficiency Syndrome (AIDS), can often be quite well controlled through rigorous screening of human blood products and avoidance of certain voluntary behaviors. Further, as blood exchange is typically much less common between humans than air exchange, the amount of behavior that must be controlled to reduce transmission is often vastly reduced and general “social distancing” is typically not required. However, even blood borne diseases can be hard to control because of factors such as transmission by biting insects (e.g. mosquitos) and the simple dynamics of human society which often encourage or necessitate interactions where transmission can take place.

Social distancing and masks were utilized initially to combat the spread of COVID-19 for many reasons, but one of the primary was that, especially in the early stages of the pandemic, it was difficult to determine who was infected and contagious. It was recognized fairly early that carriers could be contagious before they showed any overt symptoms of the disease. Further, many that were infected were asymptomatic over the entire course of the disease having no, or only very light, symptoms that were indistinguishable from them having benign causes. Because there was no effective way to identify who was dangerous to others, systems and methods were used to reduce risks that were vastly over inclusive. In effect, the behavior of large numbers of individuals that were not contagious was constrained in an attempt to constrain the behavior of the few individuals that were contagious simply because they could not be identified.

Further, the need to maintain the operation of essential services resulted in disparate impacts of contagion spread. “Essential workers” were often forced into situations of dramatically increased risk of exposure because they could not abide by distancing requirements. In effect, essential workers were often not protected by the limitations resulting in them having no reductions to likelihood of infection. At the same time, the over inclusive nature of distancing requirements for others resulted in the policies becoming increasingly unpopular over time with individuals actively protesting their need to distance or wear masks.

As could be predicted with a policy that was over inclusive for many while being completely ineffective for others, and which continued for a long period of time, the systems and methods did not clearly result in increased or decreased infection rates, which further drove those upset with the restrictions to further disregard them. This created a viscous cycle where the systems of distancing and masks did not stop the spread of disease while at the same time resulting in severe restrictions on personal freedoms and economic and social damage to society as a whole.

While social distancing and even quarantine could be considered to be a relatively easy solution to halt or slow disease progression on the surface, social distancing was not an easy program to implement or maintain. Humans are naturally social animals and the need to keep large numbers of individuals at home for long periods, often suddenly and often unexpectedly, caused major economic upheaval due to disruption of work and school, the true impact of which is unlikely to be understood for many years. Further, the longevity of social distancing from the COVID-19 pandemic resulted in undesired modifications in the operation of human society as well as increased anxiety and isolation. The full amount of damage done to the fabric of human society by extended lockdowns may never actually be known.

It should be recognized that at the start of the COVID-19 pandemic, quarantine was one of the few tools that was even available and could be deployed with sufficient speed to deal with pandemic conditions which could have resulted in exponential infection rate growth and failure of critical infrastructure to continue to respond to disease conditions. As those infected with COVID-19 were believed able to transmit the disease many days prior to exhibiting symptoms, every person was a potential carrier and there was no way to limit protective measures only to those that were infected. Further, as many individuals never exhibited symptoms at all, it made it impossible to identify them as potential vectors for transmission. What was needed over time, however, was widespread rapid testing as this would allow those who were dangerous to others to be distanced, while allowing those that were not to maintain economic and social infrastructure.

While tests to definitively determine if someone had COVID-19 were developed very quickly, they were in short supply due to them requiring numerous specialized materials. Further, even when tests became generally available, they typically had a relatively long waiting period between the test being taken and when definitive results could come back. This meant that the number of people who had COVID-19 via such tests was identifiable only as a past time-period determination which, in turn, made it difficult to plan for the future at any given time. It also resulted in a need to quarantine individuals who, in the end, were not actually sick but who, for a period of time, could not be found to be safe. Thus, initial available testing, while useful to track infection spread and related occurrences, was near useless as a tool to inhibit further transmission.

It was asserted relatively early on during the COVID-19 pandemic that what was needed to really control the pandemic and halt further spread was an easy test that produced rapid results to determine if a person had COVID-19 or, more accurately, was able to transmit COVID-19 to others. Such a test would allow contagious individuals to be identified and they could be segregated from those who were not infected (either voluntarily or not). While a number of rapid in home tests were developed and eventually deployed, they all lacked the capability of being performed on the highly regular basis that was really desired. These tests, while relatively inexpensive individually, did consume resources every time they were performed. Because of this, they were still somewhat difficult for anyone to perform on a consistent and pervasive schedule (e.g. once a day). Instead, these tests were often only used when infection chance was dramatically increased (e.g. due to a possible exposure) and easy tests that could be performed quickly and pervasively, such as the taking of body temperature, but which may have had limited actual value in detecting if an individual had COVID-19 or was contagious, saw rapid and pervasive adoption.

Specifically, guidelines for interacting with others during the pandemic often required or suggested that an individual would need to have a normal (e.g. not elevated) body temperature to attend group events. For example, individuals such as children attending school or camp or adults entering workplaces often had to have their temperature taken, or to assert that they had taken it and it was normal, before entering and being allowed to interact with others. This often occurred even when those interactions were still limited by masks and distancing. All this temperature taking was also still done even when evidence began to show that temperature was likely a lagging indicator of COVID-19 infection. That is, a normal temperature was still used to allow a user to interact with others where infection of others was more likely by them if they were contagious even after it had been determined that they could be contagious even without an increased temperature.

A normal body temperature was used to clear users as safe, however, because it was easy to obtain. The thermometer has existed for decades and has been an indispensable tool for detecting disease. It is well recognized that when a human's immune system is highly active, the body temperature rises. This rise can be detected in a few seconds by modern infra-red (IR) thermometers without any substantial invasion of personal space or rights. Further, an individual test using an IR thermometer is essentially free (once the thermometer itself was purchased) and it is easy for single IR thermometer with just one set of batteries to perform hundreds if not thousands of tests. This is why the taking of temperature before participating in any form of gathering became pervasive in many situations during the COVID-19 pandemic. As a mechanical and physical test, it was much simpler and faster than any chemical or biological test (which could more definitively identify COVID-19) could ever be.

However, the problem with using body temperature as indicative of disease state is that body temperature typically only increases after the immune system is highly active. Thus, an individual often “feels sick” before their temperature actually registers as increased. It is for this reason that body temperature, until the COVID-19 pandemic, was not broadly used to inhibit disease transmission. Instead, body temperature was only commonly used to detect disease in infants and young children who often could not communicate specifics of how they felt sick (only that they were uncomfortable) or to verify that an individual who had an incentive to stay home sick, or to gather with others even when they clearly were sick, was more correctly identified. In effect, before COVID-19, many people had incentive to lie about being sick (or not) and body temperature could be used as a tool to detect such lies. During COVID-19, people generally lost such incentive to lie, but body temperature began to be used more pervasively for verification.

Given the highly contagious nature of diseases that utilize airborne transmission such as COVID-19, the risk of another pandemic should not be trifled with. Further, even without the emergence of another novel pathogen of the same transmissibility and danger, the human race has had to deal with negative effects of airborne pathogens for millennia. The common cold and the flu are all viruses that also utilize airborne transmission and while they typically do not cause global upheaval on an ongoing basis, they do cause large amounts of suffering, hospitalization, and even death every year. Further, while they typically do not cause the level of economic disruption of COVID-19, flu and other respiratory diseases cause economic loss every year.

No two viruses or pathogens are the same and the ability of viruses to mutate can result in an ongoing battle to locate, identify, and destroy them. However, many viruses that afflict humans, and particularly those that utilize airborne transmission, are dangerous not due to the pathogen directly, but that the viruses commonly get into the human respiratory system or other essential system. The human body's response to the virus then often results in problems that are what actually causes permanent injury or death to the individual. For example, for respiratory ailments, respiratory distress including the generation of excess mucus in the respiratory system along with inflammation which can lead to coughing, nasal symptoms such as congestion (rhinitis) and runny nose (rhinorrhea), headaches, and general weakness from the body's reduced capacity to handle air. In other diseases, the virus response can result in damage to blood cells which can in turn result in damage to blood vessels and heart tissue.

In most originally healthy people, the response to most viruses is not so sufficient as to be dangerous, but for those with certain pre-exiting conditions, respiratory and cardiac symptoms of virus infection can require hospitalization and mechanical intervention. These can allow the person to live through the time they are afflicted by the disease by artificially bypassing the natural human processes which are afflicted. However, hospitalization presents increased risk that the patient can expose others to additional pathogens and hospital intervention presents their own risks as well.

Outside of infection, degenerative illness, acute conditions, and simple aging can also cause changes to the operation of the body which can result in both acute dangers or long term degeneration in similar fashion to acute illness. For example, heart attacks result in the heart stopping which can result in death if not detected quickly. While heart attacks are an acute condition with immediate concern, the causes of heart attacks are often degenerative and take many months if not years of accumulation before the danger is realized. Similarly, degenerative illnesses such as Amyotrophic Lateral Sclerosis (ALS) can cause loss of motor control which can result in trouble breathing due to inability to accurately control the lungs.

### SUMMARY OF THE INVENTION

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, it is desired to have systems and methods which can produce a predictive test whose purpose is to detect modification to respiration or cardiac function. The systems and methods are preferably mechanical, as opposed to chemical or biological, so that performance of each individual test consumes little to no resources, returns a very quick output, and is generally non-invasive.

Described herein are systems and methods for the physical detection of respiration and cardiac function and particularly alteration in respiration which can be used to detect potential damage to the lungs, mucus accretion, or other symptoms which can be indicative of respiratory infection. This physical detection can be performed using specialized simple sensors which are included as part of a bed or are attached to or placed on or under a mattress or, in an embodiment, using physical sensors in mobile devices such as smart phones allowing for much broader data collection, monitoring, and identification of those afflicted.

There is described herein, among other things, a method for identifying a user as contagious to others, the method comprising: instructing said user to obtain a waveform indicative of their respiration; said user obtaining said waveform using a mobile communication device by: placing said mobile communication device, which includes an accelerometer, against their chest or abdomen; activating a software application on said mobile communication device, said software application using said accelerometer to generate said waveform; comparing said waveform to other waveforms; detecting a presence of an anomaly in said user's waveform which was previously indicative of other users being contagious to others; and identifying said user as contagious based on said presence of said anomaly.

In an embodiment of the method, the mobile communication device comprises a smartphone.

In an embodiment of the method, the mobile communication device transmits said waveform to a central server for said comparing.

In an embodiment of the method, the comparing includes comparing said waveform to waveforms generated from other users.

In an embodiment of the method, the mobile communication device is held in place against said chest or abdomen by gravity.

In an embodiment of the method, the mobile communication device is held in place against said chest or abdomen by said user's hand.

In an embodiment of the method, the mobile communication device is held in place against said chest or abdomen by a strap or band.

There is also described herein, in an embodiment, a method for identifying a user as not contagious to others, the method comprising: instructing said user to obtain a waveform indicative of their respiration; said user obtaining said waveform using a personal mobile communication device by: placing said mobile communication device, which includes an accelerometer, on their chest or abdomen; activating a software application on said mobile communication device, said software application using said accelerometer to generate said waveform; comparing said waveform to prior waveforms from said user; detecting that said user's waveform matches said prior waveforms; and identifying said user as not contagious based on said matching.

In an embodiment of the method, the mobile communication device comprises a smartphone.

In an embodiment of the method, the mobile communication device transmits said waveform to a central server for said comparing.

In an embodiment of the method, the comparing includes comparing said waveform to waveforms generated from other users.

In an embodiment of the method, the mobile communication device is held in place against said chest or abdomen by gravity.

In an embodiment of the method, the mobile communication device is held in place against said chest or abdomen by said user's hand.

In an embodiment of the method, the mobile communication device is held in place against said chest or abdomen by a strap or band.

There is also described herein, in an embodiment, method for monitoring a disease state of a user, the method comprising: said user having a mobile communication device including an accelerometer; instructing said user to obtain a waveform indicative of their respiration; said user obtaining said waveform using said mobile communication device by: placing said mobile communication device against their chest or abdomen; activating a software application on said mobile communication device, said software application using said accelerometer to generate said waveform; comparing said waveform to prior waveforms from said user; and tracking said disease state via changes to said waveforms over time.

In an embodiment of the method, the mobile communication device comprises a smartphone.

In an embodiment of the method, the mobile communication device transmits said waveform to a central server for said comparing.

In an embodiment of the method, the comparing includes comparing said waveform to waveforms generated from other users.

In an embodiment of the method, the mobile communication device is held in place against said chest or abdomen by gravity.

In an embodiment of the method, the mobile communication device is held in place against said chest or abdomen by said user's hand.

In an embodiment of the method, the mobile communication device is held in place against said chest or abdomen by a strap or band.

### BRIEF DESCRIPTION OF THE DRAWINGS

FIG. 1 depicts a general overview of an embodiment of a system for obtaining and monitoring respiration, cardiac function, and other health data from physical input.

FIG. 2 shows a first embodiment of a sensor which is suitable for inclusion within, on or under a mattress or chair cushion.

FIG. 3 shows a second embodiment of a sensor which is suitable for inclusion within, on or under a mattress or chair cushion.

### DETAILED DESCRIPTION OF PREFERRED EMBODIMENTS

Throughout this disclosure, the term “physical detection” or “mechanical detection” is used to broadly refer to technologies which detect changes in the physical world as opposed to chemical detection or biological detection. Physical detection in this disclosure will commonly utilize changes in movement (including starting, stopping, direction, acceleration, or velocity), or changes in orientation. Physical detection also includes changes to electrical or magnetic fields as well as alteration of structural processes and subatomic forces. In many respects, physical detection relates to detection of any change within electromechanical parameters in the operation of the human body as opposed to biological or chemical changes (although such biological and chemical changes are recognized as often causing the changes in electromechanical parameters). Physical detection can be carried out by a wide variety of instruments, but devices such as motion detectors (including cameras in all electromagnetic spectrums), accelerometers, magnetometers, gyroscopes, and other types of well-known measurement devices are all capable of physical detection. Physical parameters including, but not limited to, acceleration, g-force, angular velocity or change in magnetic field can all be measured.

The term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, cell phones, mobile phones, smart phones, tablet computers, server farms, hardware appliances, minicomputers, mainframe computers, video game consoles, handheld video game products, and wearable computing devices including, but not limited to eyewear, wristwear, pendants, fabrics, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the term “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service (SAAS), or other distributed or collaborative computer networks.

Those of ordinary skill in the art also appreciate that some devices which are not conventionally thought of as “computers,” nevertheless exhibit the characteristics of a “computer” in certain contexts. Where such a device is performing the functions of a “computer” as described herein, the term “computer” includes such devices to that extent. Devices of this type include, but are not limited to: network hardware, print servers, file servers, NAS and SAN, load balancers, and any other hardware capable of interacting with the systems and methods described herein in the matter of a conventional “computer.”

Throughout this disclosure, the term “software” refers to code objects, program logic, command structures, data structures and definitions, source code, executable and/or binary files, machine code, object code, compiled libraries, implementations, algorithms, libraries, or any instruction or set of instructions capable of being executed by a computer processor, or capable of being converted into a form capable of being executed by a computer processor, including, without limitation, virtual processors, or by the use of run-time environments, virtual machines, and/or interpreters. Those of ordinary skill in the art recognize that software can be wired or embedded into hardware, including, without limitation, onto a microchip, and still be considered “software” within the meaning of this disclosure. For purposes of this disclosure, software includes, without limitation: instructions stored or storable in hard drives, RAM, ROM, flash memory BIOS, CMOS, mother and daughter board circuitry, hardware controllers, USB controllers or hosts, peripheral devices and controllers, video cards, audio controllers, network cards, Bluetooth® and other wireless communication devices, virtual memory, storage devices and associated controllers, firmware, and device drivers. The systems and methods described here are contemplated to use computers and computer software typically stored in a computer- or machine-readable storage medium or memory.

Throughout this disclosure, the term “network” generally refers to a voice, data, or other telecommunications network over which computers communicate with each other. The term “server” generally refers to a computer providing a service over a network, and a “client” generally refers to a computer accessing or using a service provided by a server over a network. Those having ordinary skill in the art will appreciate that the terms “server” and “client” may refer to hardware, software, and/or a combination of hardware and software, depending on context. Those having ordinary skill in the art will further appreciate that the terms “server” and “client” may refer to endpoints of a network communication or network connection, including, but not necessarily limited to, a network socket connection. Those having ordinary skill in the art will further appreciate that a “server” may comprise a plurality of software and/or hardware servers delivering a service or set of services. Those having ordinary skill in the art will further appreciate that the term “host” may, in noun form, refer to an endpoint of a network communication or network (e.g., “a remote host”), or may, in verb form, refer to a server providing a service over a network (“hosts a website”), or an access point for a service over a network.

Throughout this disclosure, the term “transmitter” refers to equipment, or a set of equipment, having the hardware, circuitry, and/or software to generate and transmit electromagnetic waves carrying messages, signals, data, or other information. A transmitter may also comprise the componentry to receive electric signals containing such messages, signals, data, or other information, and convert them to such electromagnetic waves. The term “receiver” refers to equipment, or a set of equipment, having the hardware, circuitry, and/or software to receive such transmitted electromagnetic waves and convert them into signals, usually electrical, from which the message, signal, data, or other information may be extracted. The term “transceiver” generally refers to a device or system that comprises both a transmitter and receiver, such as, but not necessarily limited to, a two-way radio, or wireless networking router or access point. For purposes of this disclosure, all three terms should be understood as interchangeable unless otherwise indicated; for example, the term “transmitter” should be understood to imply the presence of a receiver, and the term “receiver” should be understood to imply the presence of a transmitter.

For purposes of this disclosure, there will also be significant discussion of a special type of computer referred to as a “mobile communication device” or simply “mobile device”. A mobile communication device may be, but is not limited to, a smart phone, tablet PC, e-reader, satellite navigation system (“SatNav”), fitness device (e.g. a Fitbit™ or Jawbone™) or any other type of mobile computer, whether of general or specific purpose functionality. Generally speaking, a mobile communication device is network-enabled and communicating with a server system providing services over a telecommunication or other infrastructure network. A mobile communication device is essentially a mobile computer, but one which is commonly not associated with any particular location, is also commonly carried on a user's person, and usually is in near-constant real-time communication with a network.

The system may utilize a “positioning system” which is any form of location technology and will typically be a satellite positioning system such as GPS, GLONASS, or similar technology, but may also include inertial and other positioning systems, and wireless communication to enable detection of location such as beacon technology. Any wireless methodology for transferring the location data created by the positioning system to the other component parts of the system to which it is communicatively networked is contemplated. Thus, contemplated wireless technologies include, but are not limited to, telemetry control, radio frequency communication, microwave communication, GPS and infrared short-range communication.

In an embodiment, the systems and methods described herein utilize sensors for physical detection to detect patterns, and particularly changes to those patterns, in human or other animal respiration and/or cardiac function that are indicative of declining or altering health or indicative of upper or lower respiratory infection, disease, allergy, viral contamination or other issues of interest. As the physical detection can be sufficiently accurate to detect small changes imperceptible to a human user (and even a trained medical professional), the detection of potential illness can take place early in an infection cycle and well in advance of the human identifying or suffering from symptoms. In an embodiment, the physical detection is specifically the movement of the human's chest as they breathe and/or their heart beats.

This disclosure will often discuss the need to detect changes in respiration in conjunction with the determination of whether or not a user is currently infected with a particular disease and, more specifically, is capable of infecting others with that disease. It should be recognized that such determination will generally be imperfect and the system cannot definitely state that any individual, at any given time, is or is not contagious to any other specific individual as too many variables go into that determination. Instead, the purpose of these systems and methods is to provide improved gatekeeping in such detection. Specifically, the systems and methods discussed herein are designed to better determine when any individual is not contagious to most people compared to existing systems which typically only test individuals that already are at increased risk of being contagious to determine if they are.

Further, while the present disclosure is also focused on the use of the systems and methods to detect transmissible infection, it should be recognized that any system which allows for more regular monitoring of human body function provides not only the ability to detect transmissible disease infection, but the ability to detect alteration of function which may be indicative of individual deterioration. Thus, while the systems and methods discussed herein provide for a number of benefits with regards to avoiding the need for broad lockdowns or gathering restrictions with regards to contagious diseases, they can also serve to provide increased data for health monitoring individually.

Detection of potential disease or deterioration using the present systems and methods can occur in privacy or isolation for the patient and with the patient remote from a health care professional. Detection may also be performed relatively non-invasively as the sensor is typically external to the body and may be placed external to clothing or other covers. As such, the data collection may be used in public areas to allow access to a gathering or other human activity where the presence of a disease state in one individual could be dangerous to others present. This type of detection can, thus, provide advance notice of a disease state and potentially provide sufficient time for appropriate evaluation, isolation, monitoring and/or medical intervention to inhibit disease transmission or progression and/or possibly before the illness produces irreversible complications. In the case of possible contamination to other individuals, measures can also be taken to limit the spread of disease and to notify those with potential contact to an infected individual early to attempt to halt further spread. This can result in increased effectiveness (and acceptance) of lockdowns, quarantines, and other isolating measures by better limiting them only to those who are an increased risk to others.

The systems and methods may be used for quick detection of infection or changed state over a short time frame. Alternatively, they may be used for monitoring of disease state over a long term. For example, long term data of patients with degenerative illness may be obtained to monitor the disease progression. Further, the systems and methods discussed herein may also detect acute changes in long term illness which could indicate an immediate health concern requiring an immediate health response to prevent permanent injury or death.

In an embodiment, the system and methods contemplated herein make use of sensors that measure readily available electromechanical changes using readily available sensors to implicate biological changes which cause those electro mechanical changes. Specifically, in an embodiment, the systems and methods herein make use of the mechanical movement of the chest and other externally visible body structures to detect the current state of the lungs and/or heart (which are not directly visible). The systems and methods will often utilize readily available accelerometers, magnetometers, gyroscopes, inclinometers, and similar sensors already present in a user's mobile device to obtain the data.

In an embodiment, the sensors may be tethered to the subject such as being placed against their chest, abdomen, or back and held in place with a strap or similar structure around their torso. They may also be simply positioned to rest on the chest, abdomen, or back utilizing gravity to hold them in place or may be held in place with a hand or similar restraint. Alternatively, the sensors may be placed on a bed or chair under a user (regardless of their orientation to the bed or chair), or may be carried by a user or otherwise held in proximity to certain points of their body. The mobile device sensor may be placed by the user for the primary purpose of detecting this body movement, or may be used to detect body movement as a secondary function. For example, a mobile device may detect respiration while the mobile device is primarily being used by the user for another purpose, for example as a digital reading device, for accessing webpages, or for playing video games.

When such a device is placed on or near the abdomen or chest, acceleration, g-force, angular velocity and magnetic field changes to the chest area, amongst other things, can be readily measured. External changes to the human condition, for example that the chest rises with inhalation and falls on exhalation, are well-known and occur not only in respiration but also with heart beats. Devices can also be placed under a human user such as on the surface of or within a mattress or chair which will have a similar result as such movement is translated via the surface into the underlying sensor.

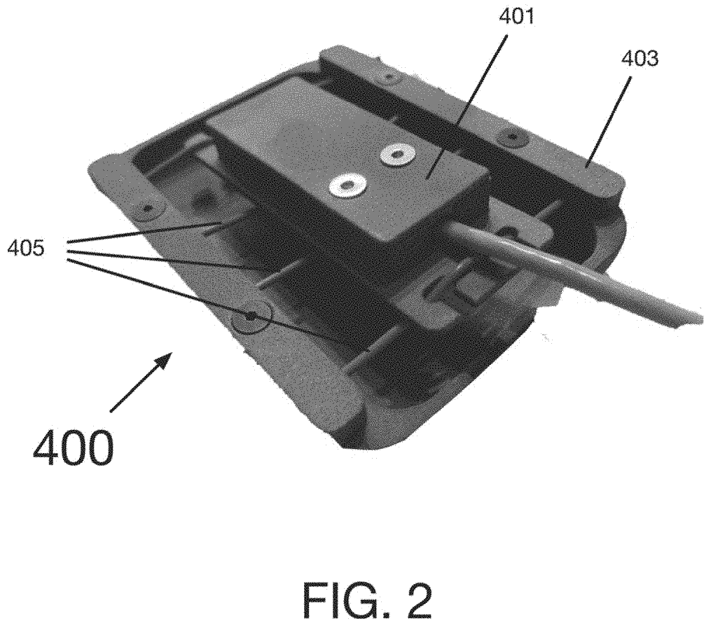

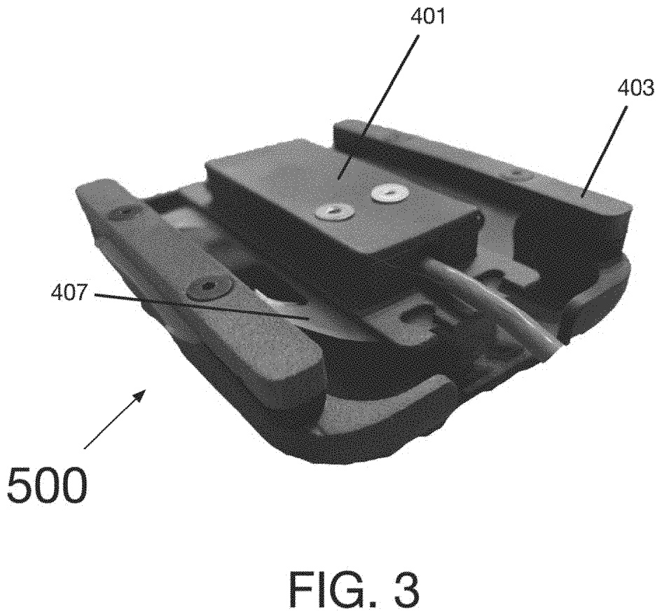

While sensors present in existing mobile devices provide an immediate and ready source of sensors, in an alternative embodiment sensors specifically designed for the purpose of measuring chest or abdomen movement, particularly of a prone or sitting patient, may be used instead of or in addition to sensors in mobile devices. Examples of two such sensors (400) and (500) are shown in FIGS. 2 and 3 and discussed in detail in U.S. Provisional Patent Application Ser. No. 63/054,078, the entire disclosure of which is herein incorporated by reference. In these Figs, an accelerometer (401) is mounted to a support (403) via a spring (405) or (407). Spring (405) simply comprises elastic bands or other supports having internal springiness. Spring (407) comprises a leaf spring or similar component interconnecting the accelerometer (401) and support (403). It would be understood by one of ordinary skill in the art that alternative spring structures including without limitation, coil or disk springs, could be used in alternative embodiments.

The construction of sensors (400) and (500) allows for the accelerometer (401) to be moved relative to the support (403) (regardless of which is against the torso of the user) while at the same time providing sufficient support for the accelerometer (401) that it can obtain a signal from the movement of the chest or abdomen. Sensors (400) and (500) would typically be placed in, on the surface of, or under a mattress or chair cushion so that when a user's mass is placed against them, the biasing of the spring (405) or (407) is partially countered by their mass. Small changes in the pressure from their chest moving (particularly if they are lying on their chest on top of the sensor (400) or (500)) will generally be readily detected as movement of the accelerometer (401) relative to the support (403) will generally require less force than for the user's breathing (or heartrate) to raise their mass from the accelerometer.

The motion of the chest or abdomen will typically be detected by the accelerometers, magnetometers, gyroscopes, and/or inclinometer present in the mobile device as a waveform or combination of waveforms. This waveform can be processed by algorithms to identify heartbeats and estimate respiration features or patterns. Certain combinations of features (for example, specific waveforms of inhalation) are then correlated to early signs of potential infection, respiratory issues or other illness. This correlation can be carried out at a macro scale level where waveforms from one specific user are compared to waveforms gathered from a large group of other users both with and without respiratory conditions of interest. Alternatively, or additionally, changes in waveforms patterns over time for each person individually (comparing current waveforms from that individual against prior waveforms from that individual) may also be correlated to signs of potential infection, respiratory issues, or other illness.

Monitoring of changes in the patterns over time typically allows for more accurate identification of potential illness (less false alarms), earlier in time, allows for more conditions to be detected and also allows for the system to adjust on a personal level for each individual. Adjusting for each individual is generally preferred as each person's “regular” condition is likely slightly different. That is, each person's physiology typically results in them generating different waveforms. However, both the features indicating illness and the deteriorating health patterns can be predetermined by experts in the field (such as pulmonologists) or learned by the system over time (such as through the use of a neural net or other “artificial intelligence” technology) for each person or for populations as a whole.

Comparison and detection may be accomplished, for example, by establishing a baseline set of features and parameters and monitoring for anomalies. As an example, coughing, shortness of breath (gasping breaths), number of respirations per minute, respiration variability, depth of breath, or completeness of breath could all be estimated by the waveforms of the aforementioned sensory equipment and the values or changes of values over time correlated to particular irregularities due to illness. As another example, signs of cardiovascular compensation for incomplete blood oxygenation can be identified by evaluating simultaneously changes in heart and respiratory rates or variability in heart and respiratory rates.

While many of the physical parameters of human physiological changes can already be measured by specialized equipment, such specialized equipment is often not available to a user in their home. For example, a human user may not be able to measure blood oxygenation from the light sensor typically attached to their finger simply because most humans do not have ready access to such a light sensor outside of a hospital setting. However, the ubiquity of mobile devices and their general availability to human users can allow for broad detection of breathing waveforms and quick evaluation of new data. Further, the systems and methods discussed herein can detect the underlying causes of reduced blood oxygenation and not just that it is occurring.

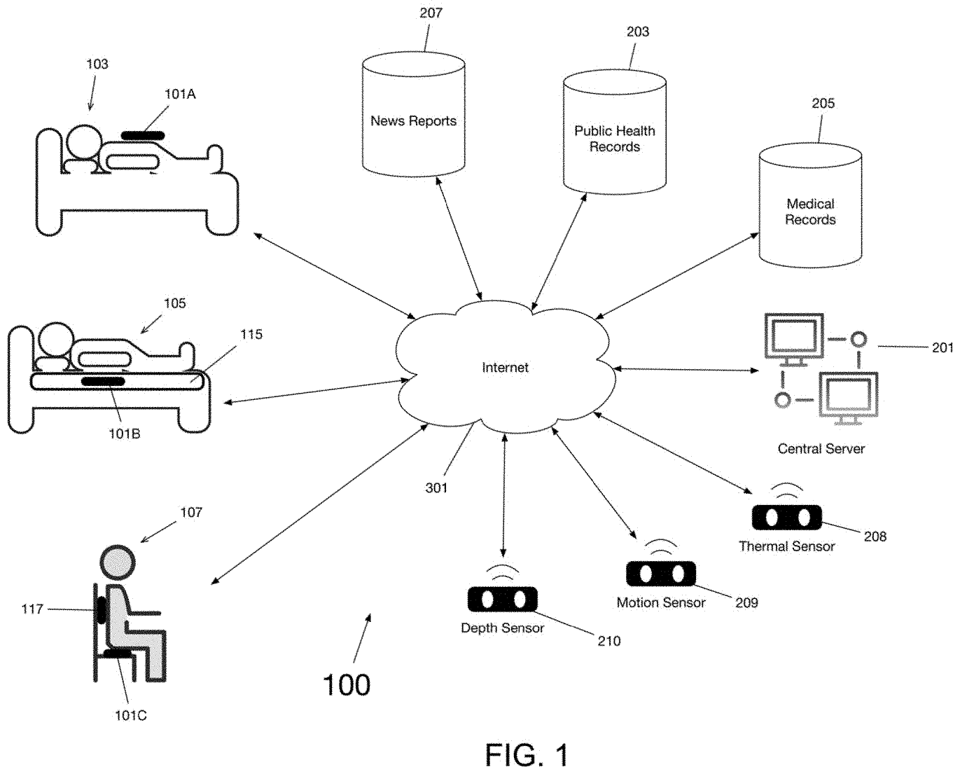

FIG. 1 illustrates an embodiment of a system for collating respiration information from any number of users including from a single user. Human users (103), (105), and/or (107) would activate software (a software application or “app”) on an associated mobile device (101A), (101B), or (101C). The activation may be singular or may occur at multiple times preferably in a common place and under common circumstances. For example, the user (103) may be requested to activate the software when they wake up and/or before they go to bed every day. This allows for the collection of large numbers of data points which can be used to determine that user's “normal” parameters and better detect deviation therefrom. The user (103) could then be instructed to lie flat and place their mobile device (101A) on their chest or abdomen at a specific location as illustrated by user (103). The mobile device (101A) could sense strength of heartbeat or other factors of the user to determine if the location is as desired for measurement. Alternatively, instead of placing the mobile device (101A) on themselves, they may simply hold it up solidly against their chest or it may be attached to their chest such as via a strap or band so it can be held in place without them interacting with it.

Alternatively, the mobile device (101B) may be placed on, under, or within the mattress (115) of a patient (105). Patient (105) may be a resident of a nursing home or similar facility who is unable to leave bed without assistance. Alternatively, the mobile device (101B) may be placed by a user under, on, or within their mattress before going to sleep. Positioning on the mattress may be virtually anywhere and can be on the top, bottom, or any side of the mattress. The device (101B) could also, in another embodiment, be placed on other components of the bed including the frame, box spring, headboard, or footboard if the user is expected to directly or indirectly cause the device to move relative to them. The device (101B) may operate according to a set time schedule or upon detection of certain conditions (e.g. that the user has entered REM sleep) instead of when activated by the user. A still further mobile device (101C) could be placed in a chair (117) in the room of a patient (107) in a hospital isolation ward, or simply in a waiting room chair before entering a doctor's office or restaurant and simply activated by pressure when they sat down. Mobile device (101C) could be two linked devices as shown, or could include an externally connected sensor (such as, but not limited to those shown in FIGS. 2 and 3) to provide different monitoring locations for a single user (103).

The user (103) may be passively monitored or may be instructed to breathe normally or to carry out a specific series of breathing exercises such as specifically taking the deepest breaths possible or to breathe and hold their breath for a period of time. They could also be asked specifically to cough or to move in a particular way during testing. The users (105) and (107) may also be similarly instructed or may be passively monitored.

Regardless of the nature of the user's (103), (105), or (107) breathing or actions, the mobile device (101A), (101B), or (101C) would then record the data from the sensors in the mobile device (101A), (101B) or (101C) related to the chest and/or abdominal movement of the user (103), (105), or (107) during these actions. This data will typically correspond to the physical displacement of the chest or abdomen during the actions (typically relative to the user, but motion in space may also be used), the velocity of the chest or abdominal movement, and/or the acceleration of the chest or abdomen during the movement. In many cases, the collected data will be provided as a waveform and the data may be processed, such as through the use of noise cancellation or other actions, to clean up data collection as is understood by those of ordinary skill in the art.

Once collected, the data could then be stored by the app and compared to prior data recorded by the app or which has been supplied to the memory of the mobile device (101) from elsewhere. The comparison will typically be looking for anomalies in the data which are indicative of a change from normal. For example, if breathing was found to be substantially shallower (e.g. the waveform had reduced magnitude compared to prior waveforms) that could be used to indicate that a user may not be taking in as much oxygen. Similarly, a user may take longer to complete a deep breath if they are having trouble getting as much air into their lungs which may be the case if their throat is constricted due to an allergic reaction, for example. The identification of any anomaly compared to a “normal” waveform of breathing for this user will typically be indicative of a potential concern.

When an anomaly is detected, it will typically be the case that the nature of the anomaly be analyzed and it be determined if the specific anomaly corresponds to what is expected from a particular change in body function. In some cases, the anomaly may be of a known type. For example, a user with asthma or allergies, for example, may have an anomaly where their waveform changes from normal to a waveform corresponding to those recorded for this user during a prior asthma attack or allergic reaction. The detection of this anomaly may be used to indicate to the user that such condition is likely and that they should utilize medications or other interventions (such as an asthma inhaler). Upon doing so, the user could be retested to see if that has resolved the anomaly.

In some cases, such as with communicable disease, the individual user may be breathing in a way that does not correspond to prior patterns for that user. For example, a user's first experience with a particular respiratory infection (e.g. COVID-19) will typically result in a breathing pattern that they may not have had before. When a previously unidentified anomaly is detected for that user, the app may share the information using a transmitter or similar device to a server (201) via the Internet (301) or another network. The data may be packaged with other data from the mobile device (101) such as time or location information or information from other apps such as fitness or health tracking software. A user (103) or a caregiver for a user (105) or (107) may also be specifically requested to enter additional information such as their current health state, if they had difficulty performing any of the breathing exercises, or if they believe they may have been exposed to a certain illness since the last time they utilized the app. This user-supplied data can also be combined with the collected motion data.

Data from the mobile device (101) may also be combined with data from other monitoring systems such as, but not limited to, systems in the room for monitoring the patient for other conditions. For example, thermal sensors (208) may be used to make sure that the data obtained by the mobile device (101) appears to be from a human patient in the room and not a different signal. This is similar to how such thermal signals may be used to detect a human user for fall detection as discussed in U.S. Pat. No. 10,453,202, the entire disclosure of which is herein incorporated by reference. Thermal sensors (208) may also be used to determine if a patient is feverish in an embodiment such as is described in United States Patent Application Publications 2008/0154138, 2007/0153871, and 2016/0150976, the entire disclosure of all of which is herein incorporated by reference.

Similarly, monitoring systems such as motion sensor (209) and depth sensor (210) which may be in the room for fall detection such as is described in U.S. Pat. Nos. 8,890,937; 9,408,561; 9,597,016; 10,080,513; 10,188,295; and 10,206,630, the entire disclosures of all of which are herein incorporated by reference, may also supply data on the patient which may be combined with the data from the mobile device (101). The data from a bed sensor (such as, but not limited to, sensor (400) or (500)) or data from any other sensor obtaining information about the patient may also be included with the data collected above. Any and all data may be sent to the server (201) encrypted and/or anonymized to protect privacy of the user (103), (105) or (107).

The central server (201) may receive the data from a plurality of mobile devices (101A), (101B), and (101C) and may collate and analyze the data from this plurality for patterns and correlations. If certain patterns are detected across users (103), (105), and (107), this may be combined with general information available from databases (203), (205), and/or (207) to which the server has access to search for patterns. For example, if the server (201) received information from multiple mobile devices in location A which appeared similarly anomalous, the server (201) may look to public health databases (203) or news databases (207) to determine if there may be an outbreak of a particular illness (such as, but not limited to, flu or SARS) reported in location A. The presence of such a possible cause may provide a first indication of correlation between the detected anomaly and the illness, for example.

The server (201) may also obtain specific health related information related to the patterns. For example, information collected by other monitors (such as, for example an electrocardiogram, blood oxygenation reading, diagnosis, and eventual outcome) could be obtained from (typically anonymized) medical records (205) of a patient that was hospitalized for a certain condition after showing similar pattern changes in breathing. Thus, if patient (105) showed a similar anomaly in their breathing pattern to patient (107) after patient (107) was hospitalized for carbon monoxide poisoning, the server (201) may attempt to determine if patient (105) had any test results in their data that would also be indicative or carbon monoxide poisoning.

If certain elements of the data received from the mobile device (101) of any user are determined to potentially be indicative of a certain illness or condition due to similarity with changes in respiration noted for other users that were previously identified as having that condition, the system (100) may notify the user of the mobile device (101) that such potential correlation has been found and suggest they consult a health professional to verify the findings. This may include performance of additional tests or even rapid treatment if the condition is sufficiently dire. The data may additionally or alternatively be passed to a health professional or caregiver that the user may have already indicated as authorized to receive their information. This person could then evaluate the data and conclusion and potentially contact the user directly, or institute care or monitoring, if they deemed such actions relevant.

As the server (201) can collect data from multiple users, the server (201) can also be used to collect epidemiological information related to those users in order to model larger scale disease issues. Specifically, the server (201) can be used to track the progression of a particular respiratory ailment through the population by recording the location and other desired characteristics within the data when symptoms of that condition are identified. This can be used both for acute infectious diseases (e.g. to track the spread of a disease such as the various forms of COVID-19 or a particular flu strain) or to track longer-term issues (such as the emergence of asthma at increased rates within certain communities).

It should be recognized that while the regular and pervasive monitoring of respiratory or cardiac function including, but not limited to, the monitoring of respiration rate, respiration variability, heart rate, heart rate variability, and other variables can be very valuable in monitoring the progression of long term disease, the use of a mobile device app in the quick collection of data which is indicative of potential infection, and more particularly indicative of the lack of potential infection, is particularly valuable to maintain or restore normalcy in human behavior during or after an infection outbreak. For example, should an outbreak of a contagious respiratory infection be detected in a particular community, individuals in that community could be instructed to test themselves at specific intervals using this system (even if they have not previously used it) before continuing with activities that may result in disease spread. In effect, the system (100) could replace less efficient testing systems such as temperature taking or the answering of survey questions before allowing a user to participate in a group activity which could increase infection in the community (increase “R-value”). Further, the system can even be used to determine how and when such testing would need to be performed.

For example, if a novel respiratory infection is believed to have entered a community, individuals may be instructed to, if possible, perform a check on their respiratory function immediately upon getting up in the morning. At the start, the server (201) will typically have limited information to associate any particular detected anomaly with an indication of any user in the community having been infected. However, the system may be able to use prior information obtained from other communities where individual data was collected from users which are known to have been infected (or known to have not been) is available. From this information, higher risk individuals may be initially detected and instructed to isolate. If infection increases among the population of the community even with the initial isolation, a pattern of respiration changes should become detectable which indicates respiration changes as the disease progresses. Basically, as members of the community who are infected become known, the knowledge of their infection can be used to compare their data to attempt to locate patterns that would be indicative of their infection.

Once a pattern indicative of infection is identified for this community, the server (201) may use software (such as a neural network or other artificial intelligence (AI) system) to go back and look for this particular pattern in the other data in this community. Detection of the pattern in others in the community can be used to direct quarantine or lockdown instructions only to those with the pattern or to those known to have contacted those with the pattern based on data about location and interaction. The patterns of both those so identified (and those not identified) can continue to be monitored and response geared as the system (100) learns to better identify patterns indicative of an individual being contagious. Thus, the system can be used to identify when an individual is potentially infected (and how their respiration and/or cardiac function alters at that time), when they are potentially contagious to others (and how their respiration and/or cardiac function alters at that time), and when other symptoms of infection can be identified (and how their respiration and/or cardiac function alters at that time). Based on these determinations, the server (201) can then begin to identify those who are more likely to be infected and/or contagious in the future. When these users (103). (105), or (107) are identified, they can be notified that they should quarantine from others or should seek medical intervention.

Even more important than identifying those that are contagious, however, is the ability to potentially eliminate those that are not contagious. Such identification can allow those that are deemed to be of low or no danger of infecting others to live and operate normally. This can allow economic and other systems to operate more efficiently during a disease outbreak than they would under more rigorous lockdown conditions. For example, children in school may be tested every morning before going to the school building or classes. Those that are cleared as non-contagious may proceed with classwork as normal. Those that may be contagious may be asked to stay home and attend classes using virtual attendance technologies until they are cleared. In this way, classes can continue without as substantial interruption as is necessary to distance an entire class when only one or two individuals may actually be contagious.

This is also a useful criteria when evaluating long term disease state. While it is useful to determine that an individual's disease state has changed or progressed, it is often even more useful to determine that it has not. A lack of progression in a progressive disease will typically indicate that a specific treatment regimen is currently effective and, therefore, should be continued and not altered. Thus, the ability to quickly, effectively, and very cheaply monitor respiration and cardiac function can provide for better understanding of the effectiveness of treatments of ailments.

While the invention has been disclosed in conjunction with a description of certain embodiments, including those that are currently believed to be useful embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

The qualifier “generally,” and similar qualifiers as used in the present case, would be understood by one of ordinary skill in the art to accommodate recognizable attempts to conform a device to the qualified term, which may nevertheless fall short of doing so. This is because terms such as “spherical” are purely geometric constructs and no real-world component or relationship is truly “spherical” in the geometric sense. Variations from geometric and mathematical descriptions are unavoidable due to, among other things, manufacturing tolerances resulting in shape variations, defects and imperfections, non-uniform thermal expansion, and natural wear. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal geometric meaning of the term in view of these and other considerations.

1. A method for identifying a user as contagious to others, the method comprising:
instructing said user to obtain a waveform indicative of their respiration;
said user obtaining said waveform using a mobile communication device by:
placing said mobile communication device, which includes an accelerometer, against their chest or abdomen;
activating a software application on said mobile communication device, said software application using said accelerometer to generate said waveform;
comparing said waveform to other waveforms;
detecting a presence of an anomaly in said user's waveform which was previously indicative of other users being contagious to others; and
identifying said user as contagious based on said presence of said anomaly.

2. The method of claim 1 wherein said mobile communication device comprises a smartphone.

3. The method of claim 1 wherein said mobile communication device transmits said waveform to a central server for said comparing.

4. The method of claim 3 wherein said comparing includes comparing said waveform to waveforms generated from other users.

5. The method of claim 1 wherein said mobile communication device is held in place against said chest or abdomen by gravity.

6. The method of claim 1 wherein said mobile communication device is held in place against said chest or abdomen by said user's hand.

7. The method of claim 1 wherein said mobile communication device is held in place against said chest or abdomen by a strap or band.

8. A method for identifying a user as not contagious to others, the method comprising:
instructing said user to obtain a waveform indicative of their respiration;
said user obtaining said waveform using a personal mobile communication device by:
placing said mobile communication device, which includes an accelerometer, on their chest or abdomen;
activating a software application on said mobile communication device, said software application using said accelerometer to generate said waveform;
comparing said waveform to prior waveforms from said user;
detecting that said user's waveform matches said prior waveforms; and
identifying said user as not contagious based on said matching.

9. The method of claim 8 wherein said mobile communication device comprises a smartphone.

10. The method of claim 8 wherein said mobile communication device transmits said waveform to a central server for said comparing.

11. The method of claim 10 wherein said comparing includes comparing said waveform to waveforms generated from other users.

12. The method of claim 8 wherein said mobile communication device is held in place against said chest or abdomen by gravity.

13. The method of claim 8 wherein said mobile communication device is held in place against said chest or abdomen by said user's hand.

14. The method of claim 8 wherein said mobile communication device is held in place against said chest or abdomen by a strap or band.

15. A method for monitoring a disease state of a user, the method comprising:
said user having a mobile communication device including an accelerometer;
instructing said user to obtain a waveform indicative of their respiration;
said user obtaining said waveform using said mobile communication device by:
placing said mobile communication device against their chest or abdomen;
activating a software application on said mobile communication device, said software application using said accelerometer to generate said waveform;
comparing said waveform to prior waveforms from said user; and
tracking said disease state via changes to said waveforms over time.

16. The method of claim 15 wherein said mobile communication device comprises a smartphone.

17. The method of claim 15 wherein said mobile communication device transmits said waveform to a central server for said comparing.

18. The method of claim 17 wherein said comparing includes comparing said waveform to waveforms generated from other users.

19. The method of claim 15 wherein said mobile communication device is held in place against said chest or abdomen by gravity.

20. The method of claim 15 wherein said mobile communication device is held in place against said chest or abdomen by a strap or band.
